(19) United States
(12) Patent Application Publication
(10) Pub. No.: US 2024/0399100 A1
(43) Pub. Date: Dec. 5, 2024

(54) INTRAARTICULAR POSTEROMEDIAL LANDMARK INJECTION FOR LOWER EXTREMITY ANALGESIA

(71) Applicant: Pacira Pharmaceuticals, Inc., Parsippany, NJ (US)

(72) Inventors: Stan Dysart, Marietta, GA (US); Jonathan H. Slonin, Palm City, FL (US); Jeffrey Gonzales, Parsippany, NJ (US)

(21) Appl. No.: 18/678,595

(22) Filed: May 30, 2024

Related U.S. Application Data
(60) Provisional application No. 63/470,231, filed on Jun. 1, 2023.

(51) Int. Cl. A61M 19/00; A61K 9/127; A61K 31/445; A61M 5/32

(52) U.S. Cl. CPC A61M 19/00; A61K 9/1271; A61K 31/445; A61M 5/32; A61M 2210/086

(57) ABSTRACT

Provided herein are methods of reducing knee pain in a subject, the method including selecting an entry point of an injection needle in a subject's leg; inserting the injection needle into the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, thereby reducing knee pain in a subject.

### CROSS-REFERENCE TO RELATED APPLICATIONS

This application claims the benefit of U.S. Provisional Application No. 63/470,231, filed Jun. 1, 2023, which is incorporated by reference herein in its entirety.

### BACKGROUND

The Adductor Canal Block (ACB) has replaced the traditional femoral nerve block, by targeting terminal branches of the femoral nerve (i.e. saphenous nerve and nerve to the vastus medialis) in order to spare quadriceps strength and facilitate early postsurgical ambulation. For total knee arthroplasty, an ACB is often combined with additional blocks in the posterior capsule to provide posterior analgesia as well. These blocks are typically performed via ultrasound guidance by a regional anesthesiologist prior to surgery. However, ultrasound approaches may not be an accessible modality for all orthopedic practices. Thus, additional block techniques are needed in order to better serve patients needing lower extremity post-operative pain management.

### SUMMARY

Provided herein are methods of reducing knee pain in a subject, the method comprising: selecting an entry point of an injection needle in a subject's leg; inserting the injection needle into the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, thereby reducing knee pain in a subject.

In some embodiments, the pharmaceutical composition comprises multivesicular liposomes. In some embodiments, the multivesicular liposomes comprise: bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and e) removing the organic solvent from the solvent spherules to form multivesicular liposomes encapsulating bupivacaine phosphate. In some embodiments, the injection needle is 1.5 inches long. In some embodiments, the injection needle tip is advanced a distance of about 1.5 inches along the angle. In some embodiments, the injection needle is an 18 gauge bevel needle. In some embodiments, selecting an entry point comprises palpating the adductor tubercle. In some embodiments, the entry point is approximately 1 cm from the adductor tubercle at the proximal border of the adjacent femoral condyle. In some embodiments, the administering comprises administering about 20 mL of the pharmaceutical composition in a single bolus. In some embodiments, the administering comprises administering about 20 mL of the pharmaceutical composition in a split bolus. In some embodiments, the split bolus comprises two boluses wherein each bolus is administered without repositioning the injection needle. In some embodiments, the split bolus comprises administering approximately a 10 mL bolus of the pharmaceutical composition, followed by a rest period, followed by approximately a 10 mL bolus of the pharmaceutical composition. In some embodiments, the methods further comprises performing a knee arthrotomy to expose the knee capsule prior to selecting the entry point of the injection needle. In some embodiments, the pharmaceutical composition spreads to affect two or more of: the saphenous nerve, the superficial branch of the saphenous nerve, the nerve to the vastus medialis, and the deep genicular nerves. In some embodiments, the pharmaceutical composition spreads to affect three or more of: the saphenous nerve, the superficial branch of the saphenous nerve, the nerve to the vastus medialis, and the deep genicular nerves. In some embodiments, the pharmaceutical composition spreads to affect the saphenous nerve, the superficial branch of the saphenous nerve, the nerve to the vastus medialis, and the deep genicular nerves. In some embodiments, less than 10% of the pharmaceutical composition is detectable in the adductor canal. In some embodiments, less than 10% of the pharmaceutical composition is detectable around the anterior femoral or lateral femoral cutaneous nerves.

Provided herein are methods of treating post-operative knee pain in a subject, the method comprising: selecting an entry point of an injection needle in a subject's leg; inserting the injection needle into the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced a distance between one inch and two inches along the angle, thereby treating post-operative knee pain in the subject.

Proved herein are methods of reducing knee pain in a subject, the method comprising: selecting an entry point of an injection needle in a subject's leg; inserting the injection needle into the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced a distance of about between one inch and two inches along the angle, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

Provided herein are methods of treating post-operative knee pain in a subject, the method comprising: selecting an entry point of an injection needle in a subject's leg; inserting the injection needle into the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced a distance of about between one inch and two inches along the angle, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a c-arm radiographic image, AP view, showing needle insertion proximal to the adductor tubercle and posteromedial to the femur.

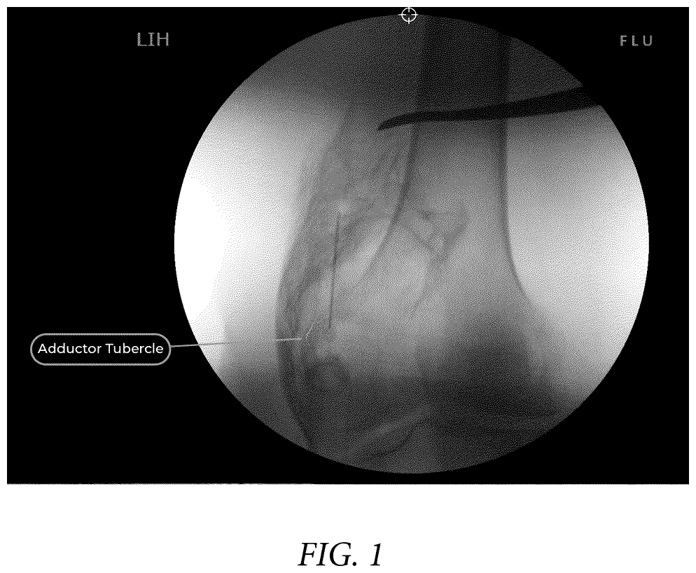

FIG. 2 is a c-arm radiographic image, lateral view, showing needle insertion proximal to the adductor tubercle and posterior to the femur, needle angle, and spread of 20 mL contrast.

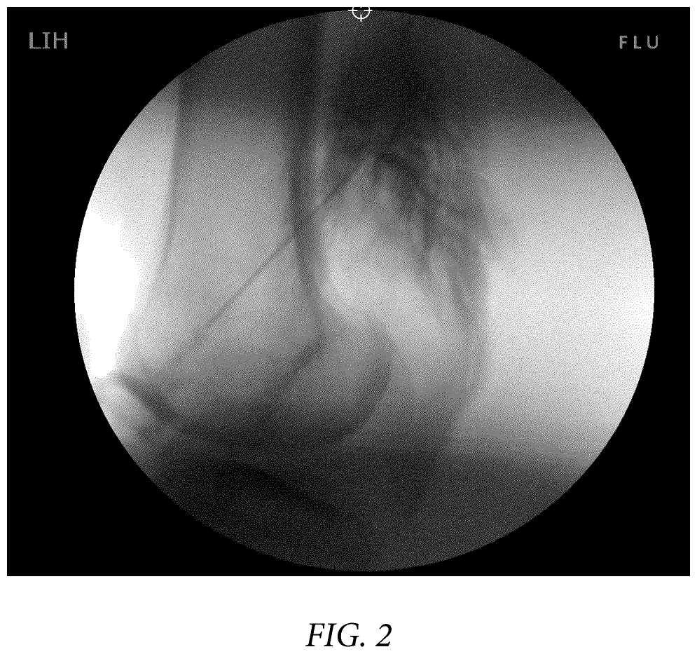

FIG. 3 is a medial view dissection image of a cadaver leg demonstrating dye spread of the intraarticular posteromedial landmark injection described herein, wherein the sartorius muscle, vastus medialis muscle, infrapatellar saphenous nerve, and adductor magnus tendon have been labeled.

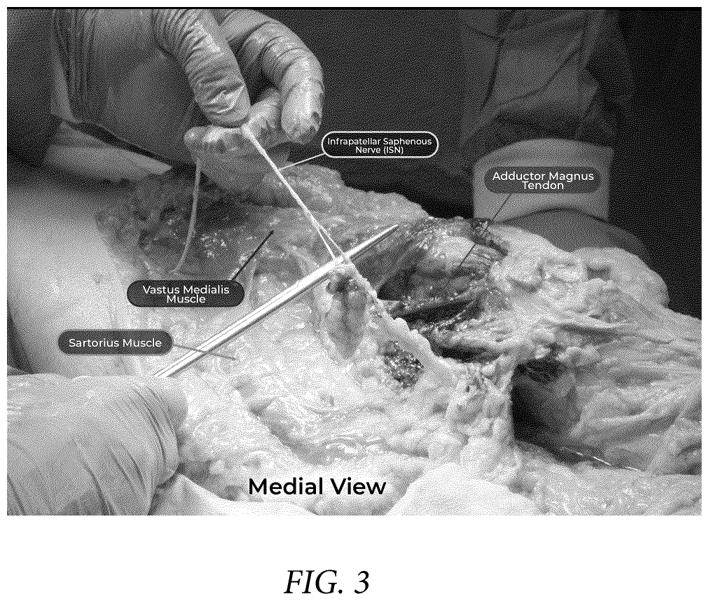

FIG. 4 is a postero-medial view dissection image of a cadaver leg demonstrating dye spread of the intraarticular posteromedial landmark injection described herein, wherein the semitendinosus tendon has been labeled.

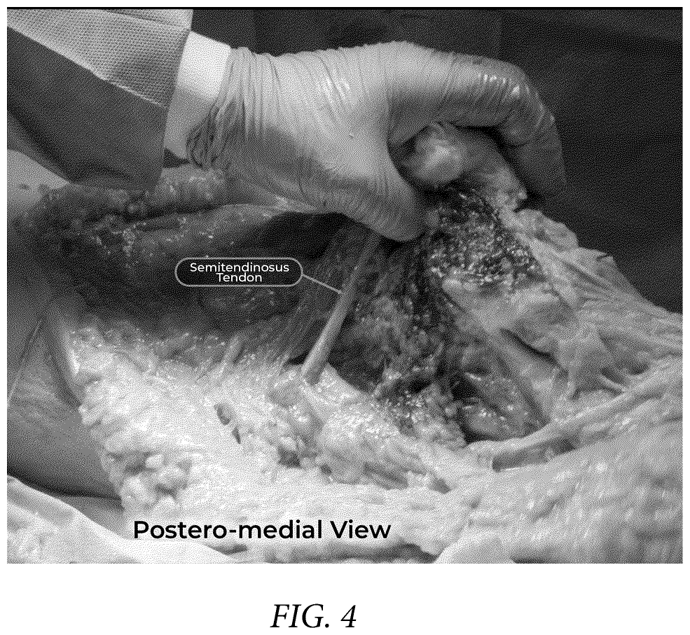

### DETAILED DESCRIPTION

Unless otherwise defined, all technical and scientific terms used herein have the same meaning as commonly understood by one of ordinary skill in the art to which this disclosure belongs. Although methods and materials similar to those described herein can be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

As used herein, the term “subject” and “patient” are used interchangeably herein in reference to a human subject.

As used herein, the term “pain” means a physiologic and/or psychologic reaction or response to a stimulus that may result in tissue damage, injury, disease, or other condition(s). Types of pain include but are not limited to acute pain, chronic pain, thermal pain, traumatic pain, chemical pain, inflammatory pain, ischemic pain, blunt pain, sharp pain, prickling pain, visceral pain, and neuropathic pain.

As used herein, the term “knee pain” is any pain associated with an injury, procedure, or disease process in the knee region. Examples of knee pain include pain associated with disease processes and inflammatory conditions and pain associated with injury or trauma, such as fractures, dislocations, tears, and sprains, such as but not limited to anterior cruciate ligament (ACL) tears and meniscus tears, pain associated with structural abnormalities including osteophytes, cartilage damage, bone marrow lesions, pain associated with knee degenerative conditions including osteoarthritis, and pain associated with surgery.

As used herein, the term “about” or “approximate” in the context of a numerical value or range means±10% of the numerical value or range recited or claimed, unless the context requires a more limited range.

### Knee Anatomy

Currently, the adductor canal block has replaced the traditional femoral nerve block by targeting terminal branches of the femoral nerve in order to spare quadriceps strength and facilitate early post-surgical ambulation. The adductor canal (subsartorial canal or Hunter's canal) is about 15 cm in length and is a narrow, fascial tunnel in the thigh. The adductor canal is located deep to the middle third of the sartorius muscle. The adductor canal provides an intermuscular passage through which the femoral vessels pass to reach the popliteal fossa, where these vessels become popliteal vessels. The adductor canal begins about 15 cm (about 6 inches) inferior to the inguinal ligament, where the sartorius muscle crosses over the adductor longus muscle. The adductor canal ends at the adductor hiatus in the tendon of the adductor magnus muscle. The saphenous nerve and, in part, the obturator nerve traverse the adductor canal. An adductor canal block typically must be performed under ultrasound guidance.

The intraarticular posteromedial landmark injection procedures described herein are performed after a medial parapatellar arthrotomy was performed wherein the adductor tubercle serves as a landmark for identifying the needle entry point to be used for the intraarticular posteromedial landmark injection procedures. The intraarticular posteromedial landmark injection procedures described herein can be performed without ultrasound guidance and solely using consistent anatomical landmarks. Thus, in some implementations the intraarticular posteromedial landmark injection procedures described herein can be performed by a surgeon without assistance from an anesthesiologist.

### Needle Entry Point

Provided herein are methods of selecting a needle entry point on a patient's leg to provide treatment of lower extremity post-operative pain. In some embodiments, the injection needle entry point is above the medial condyle at the flare of the medial condyle of the femur and approximately at the level of the adductor tubercle or the proximal most portion of the posterior femoral condyle. In some embodiments, the needle entry point is approximately a fingertip proximal to the adductor tubercle, for example, between 1 and 2 cm proximal to the adductor tubercle. In some embodiments, the needle is pointing slightly laterally, but generally parallel with the medial border of the femur.

### Needle Characteristics

Provided herein are methods of administering an injectate to a patient using a needle. In some embodiments the needle can be a Tuohy needle or a hypodermic needle. In some embodiments, the needle can be a 14 gauge, 15 gauge, 16 gauge, 17 gauge, 18 gauge, 19 gauge, 20 gauge, 21 gauge, 22 gauge, 23 gauge, or 24 gauge needle, for example, the needle can be an 18 gauge needle. In some embodiments, the needle can be a 4 inches long, 3.5 inches long, 3 inches long, 2.5 inches long, 2 inches long, 1.5 inches long, or 1 inch long, for example 1.5 inches long.

### Needle Trajectory and Injectate Administration Site

In some embodiments, the needle tip of the intraarticular posteromedial landmark injection is advanced from the needle entry point to the injectate administration site. The needle tip may be inserted between approximately 10 and approximately 20 degrees medial to the axis of the femur, including but not limited to approximately 11 degrees to approximately 19 degrees, approximately 12 degrees to approximately 18 degrees, approximately 13 degrees to approximately 17 degrees, or approximately 14 degrees to approximately 16 degrees medial to the axis of the femur, and any degrees in between. For example, the needle tip may be inserted approximately 10 degrees, approximately 11 degrees, approximately 12 degrees, approximately 13 degrees, approximately 14 degrees, approximately 15 degrees, approximately 16 degrees, approximately 17 degrees, approximately 18 degrees, approximately 19 degrees, or approximately 20 degrees medial to the axis of the femur.

Additionally, the needle tip may be inserted between approximately 40 degrees and approximately 75 degrees posterior to the femur, including but not limited to approximately 40 degrees to approximately 70 degrees, approximately 40 degrees to approximately 65 degrees, approximately 40 degrees to approximately 60 degrees, approximately 40 degrees to approximately 55 degrees, approximately 40 degrees to approximately 50 degrees, approximately 45 degrees to approximately 75 degrees, approximately 50 degrees to approximately 75 degrees, approximately 55 degrees to approximately 75 degrees, approximately 60 degrees to approximately 75 degrees, approximately 65 degrees to approximately 75 degrees, approximately 70 degrees to approximately 75 degrees, approximately 45 degrees to approximately 70 degrees, approximately 45 degrees to approximately 65 degrees, approximately 50 degrees to approximately 60 degrees, approximately 55 degrees to approximately 60 degrees posterior to the femur, and any degrees in between. For example the needle tip may be inserted between approximately 40 degrees, approximately 41 degrees, approximately 42 degrees, approximately 43 degrees, approximately 44 degrees, approximately 45 degrees, approximately 46 degrees, approximately 47 degrees, approximately 48 degrees, approximately 49 degrees, approximately 50 degrees, approximately 51 degrees, approximately 52 degrees, approximately 53 degrees, approximately 54 degrees, approximately 55 degrees, approximately 56 degrees, approximately 57 degrees, approximately 58 degrees, approximately 59 degrees, approximately 60 degrees, approximately 61 degrees, approximately 62 degrees, approximately 63 degrees, approximately 64 degrees, approximately 65 degrees, approximately 66 degrees, approximately 67 degrees, approximately 68 degrees, approximately 69 degrees, approximately 70 degrees, approximately 71 degrees, approximately 72 degrees, approximately 73 degrees, approximately 74 degrees, or approximately 75 degrees posterior to the femur.

Injection depth can be between approximately 1 inch and approximately 2.5 inches deep. For example injection depth can be approximately 1 inch, approximately 1.1 inches, approximately 1.2 inches, approximately 1.3 inches, approximately 1.4 inches, approximately 1.5 inches, approximately 1.6 inches, approximately 1.7 inches, approximately 1.8 inches, approximately 1.9 inches, approximately 2.0 inches, approximately 2.1 inches, approximately 2.2 inches, approximately 2.3 inches, approximately 2.4 inches, or approximately 2.5 inches deep. In some implementations, the injection depth can be approximately 1.5 inches deep. Different combinations of degrees medial to the axis of the femur, degrees posterior to the femur to the femur, and injection depths can be use according the disclosures herein. For example, the needle tip may be inserted at a relative angle of 20 degrees medial to the axis of the femur and 45 degrees posterior to the femur, wherein the needle is advanced to a depth of approximately 1.4 inches. As another example, the needle tip may be inserted at a relative angle of 10 degrees medial to the axis of the femur and 55 degrees posterior to the femur, wherein the needle is advanced approximately to a depth of approximately 1.5 inches. As another example, the needle tip may be inserted at a relative angle of 10 degrees medial to the axis of the femur and 70 degrees posterior to the femur, wherein the needle is advanced to a depth of approximately 1.6 inches. Other combinations of degrees medial to the axis of the femur, degrees posterior to the femur to the femur, and inches deep can be readily determined by the skilled artisan within the parameters provided above.

In some embodiments, the needle tip of the intraarticular posteromedial landmark injection at the time of pharmaceutical administration is located in the subcutaneous space superficial to the vastus medialis. In some embodiments, the needle tip of the intraarticular posteromedial landmark injection at the time of pharmaceutical administration is located proximal to the adductor tubercle intraarticularly and directed posterior and medial. In some embodiments, the needle tip of the intraarticular posteromedial landmark injection at the time of pharmaceutical administration is approximately one centimeter posterior to the posterior femoral cortex and approximately a centimeter medial to the medial femoral cortex and a centimeter proximal to the adductor tubercle. In some embodiments, the needle tip of the intraarticular posteromedial landmark injection at the time of pharmaceutical administration is located on top of the adductor tendon. In some embodiments, the needle tip of the intraarticular posteromedial landmark injection at the time of pharmaceutical administration is located anterior to the adductor tendon.

In some embodiments, the needle tip of the intraarticular posteromedial landmark injection at the time of pharmaceutical administration is directed away from blood vessels hugging the femur. The needle tip of the intraarticular posteromedial landmark injection at the time of pharmaceutical administration is not intramuscular.

### Spread of Injectate Using the Methods Described Herein

Provided herein are methods of intraarticular posteromedial landmark injection. The intraarticular posteromedial landmark injection described herein differs from an adductor canal block in at least the following ways. The injectate spread of an adductor canal block includes the adductor canal and extends to the distal end of the adductor canal. The injectate spread of an adductor canal block includes the saphenous nerve and the nerve to vastus medialis. The injectate spread of an adductor canal block does not reach the infrapatellar branch of the saphenous nerve.

By comparison to the adductor canal block, and as determined by cadaver studies described in the examples below, the intraarticular posteromedial landmark injection described herein has the spread as described infra. In some cases, the injectate of the intraarticular posteromedial landmark injection spreads between approximately 9.5 cm and approximately 15 cm, for example approximately 11.5 or 12.5 cm proximally from the joint line. The spread of the dye of the intraarticular posteromedial landmark injection methods described herein is believed to correspond to the spread of a pharmaceutical anesthetic injectate, such that the nerves and structures identified as dyed in the cadaver study described herein would be believed to be anesthetized in a living patient. Thus, such terminology as “affect”, “impact”, or “anesthetize” when describing particular nerves and/or structures are describing the anesthetic effect of the methods described herein. The skilled artisan can measure whether a particular nerve is “affected” or “impacted” using devices and/or techniques known in the art, such as a physical exam or peripheral nerve stimulator, in order to confirm whether a particular nerve has been affected by the methods described herein.

The spread of the injectate of the intraarticular posteromedial landmark injection can include subcutaneous tissue around the vastus medialis and sartorius muscles, including in the space between the vastus medialis muscle and the subcutaneous tissue and in the space between the vastus medialis and the adductor tendon. The spread of the injectate of the intraarticular posteromedial landmark injection can include the adductor tendon and the hamstrings tendon of adductor magnus. The spread of the injectate can include the adductor magnus tendon, and anterior to the adductor magnus tendon. The spread of the injectate of the intraarticular posteromedial landmark injection can include the distal portion of the infrapatellar branch of the saphenous nerve, including the superficial branch of the saphenous nerve as well as the posterior capsule. The spread of the injectate of the intraarticular posteromedial landmark injection can include the nerve to vastus medialis. The spread of the injectate can include the posterior capsule of the knee up to and including the lateral condyle of femur. The spread of the injectate of the intraarticular posteromedial landmark injection can include the area posterior to the intermuscular fascial septum that separates the anterior compartment from the medial compartment, wherein the spread is located in the medial and posterior compartments of the knee, including the deep genicular nerves. The spread of the injectate of the intraarticular posteromedial landmark injection can include the interval between the pes anserinus on Gerdy's tubercle of the tibia and the capsule, wherein the spread can include the superomedial genicular branches. The spread of the injectate of the intraarticular posteromedial landmark injection can include the area deep to the tendon of the semimembranosus.

In some embodiments, the spread of the injectate of the intraarticular posteromedial landmark injection can include spaces distal to the adductor canal, but does not include spaces within the adductor canal. In some embodiments, the spread of the injectate of an intraarticular posteromedial landmark injection is detectable in less than 30%, less than 20%, or less than 10% of the adductor canal. In some embodiments, the spread of the injectate of the intraarticular posteromedial landmark injection does not reach between the vastus medialis and sartorius muscles; in alternate embodiments, the spread of the injectate of the intraarticular posteromedial landmark injection is detectable in less than 30%, less than 20%, or less than 10% of the region between the vastus medialis and sartorius muscles. In some embodiments, the spread of the injectate of the intraarticular posteromedial landmark injection does not reach the infrapatellar saphenous, which can be superficial to the sartorius muscle; in alternate embodiments, the spread of the injectate of the intraarticular posteromedial landmark injection is detectable in less than 30%, less than 20%, or less than 10% of the region of the infrapatellar saphenous. In some embodiments, the spread of the injectate does not reach the intermuscular septum and the sartorius space; in alternate embodiments, the spread of the injectate of the intraarticular posteromedial landmark injection is detectable in less than 30%, less than 20%, or less than 10% of the region of the intermuscular septum and the sartorius space. In some embodiments, the spread of the injectate does not reach the tendon of the semimembranosus or the sciatic nerve in the region of the popliteal fossa; in alternate embodiments, the spread of the injectate of the intraarticular posteromedial landmark injection is detectable in less than 30%, less than 20%, or less than 10% of the tendon of the semimembranosus or the sciatic nerve in the region of the popliteal fossa. In some embodiments, the spread of the injectate does not reach the anterior femoral or lateral femoral cutaneous nerves; in alternate embodiments, the spread of the injectate of the intraarticular posteromedial landmark injection is detectable in less than 30%, less than 20%, or less than 10% of the anterior femoral or lateral femoral cutaneous nerves.

### Pharmaceutical Compositions

Provided herein are analgesic pharmaceutical compositions. In some embodiments the pharmaceutical compositions can be used for post-operative analgesic pain.

In some embodiments, the pharmaceutical compositions include multivesicular liposomes. Multivesicular liposomes (or “MVL”, which is used herein to refer to a multivesicular liposome or a plurality of multivesicular liposomes) are lipid vesicles having multiple non-concentric internal aqueous chambers having internal membranes distributed as a network throughout the MVL. The chambers may contain acids which are effective to enable the encapsulation of bupivacaine or a salt thereof and to modulate its release rate. A preparation of MVL is described, for example, in Kim et al., Biochim. Biophys. Acta 728, 339-348, 1983. In some embodiments, a MVL is prepared in accordance with a process as described in U.S. Pat. No. 9,192,575, incorporated by reference herein in its entirety. In some embodiments, a MVL is prepared in accordance with a process as described in U.S. Pat. No. 8,182,835, incorporated by reference herein in its entirety. In some embodiments, a MVL is prepared in accordance with a process as described in U.S. Pat. No. 8,834,921, incorporated by reference herein in its entirety. In some embodiments, a MVL is prepared in accordance with a process as described in U.S. Pat. No. 9,205,052, incorporated by reference herein in its entirety.

In some embodiments the multivesicular liposomes (“MVL”) are made by the following process. A “water-in-oil” type emulsion containing a non-hydrohalic acid salt of bupivacaine, such as bupivacaine phosphate, is formed from two immiscible phases, a lipid phase and a first aqueous phase. The lipid phase is made up of at least one amphipathic lipid and at least one neutral lipid in a volatile organic solvent. The term “amphipathic lipid” refers to molecules having a hydrophilic “head” group and a hydrophobic “tail” group and may have membrane-forming capability. As used herein, amphipathic lipids include those having a net negative charge, a net positive charge, and zwitterionic lipids (having no net charge at their isoelectric point). The term “neutral lipid” refers to oils or fats that have no vesicle-forming capability by themselves, and lack a charged or hydrophilic “head” group. Examples of neutral lipids include, but are not limited to, glycerol esters, glycol esters, tocopherol esters, sterol esters which lack a charged or hydrophilic “head” group, and alkanes and squalenes.

The amphipathic lipid is chosen from a wide range of lipids having a hydrophobic region and a hydrophilic region in the same molecule. Suitable amphipathic lipids are zwitterionic phospholipids, including phosphatidylcholines, phosphatidylethanolamines, sphingomyelins, lysophosphatidylcholines, and lysophosphatidylethanolamines. Also suitable are the anionic amphipathic phospholipids such as phosphatidylglycerols, phosphatidylserines, phosphatidylinositols, phosphatidic acids, and cardiolipins. Also suitable are the cationic amphipathic lipids such as acyl trimethylammonium propanes, diacyl dimethylammonium propanes, and stearylamines.

Suitable neutral lipids are triglycerides, propylene glycol esters, ethylene glycol esters, and squalene. Examples of triglycerides useful in the present disclosure are triolein, tripalmitolein, trimyristolein, trilinolein, tributyrin, tricaproin, tricaprylin, and tricaprin. The fatty chains in the triglycerides useful in the present disclosure can be all the same, or not all the same (mixed chain triglycerides), including all different. Both saturated and unsaturated fatty chains are useful in the present disclosure. The propylene glycol esters can be mixed diesters of caprylic and capric acids.

Many types of volatile organic solvents can be used in the present disclosure, including ethers, esters, halogenated ethers, hydrocarbons, halohydrocarbons, or Freons. For example, diethyl ether, chloroform, tetrahydrofuran, ethyl acetate, Forane, and any combinations thereof are suitable for use in making the compositions of the present disclosure.

Optionally, other components are included in the lipid phase. Among these are cholesterol or plant sterols.

The first aqueous phase includes bupivacaine or a salt thereof, such as bupivacaine phosphate, at least one polyhydroxy carboxylic acid, and at least one di- or tri-protic mineral acid. In some embodiments, also included is hydrochloric acid. The di- or tri-protic mineral acids include sulfuric acid, and phosphoric acid. Also included in the first aqueous phase are such polyhydroxy carboxylic acids as glucuronic acid, gluconic acid, and tartaric acid. The di- and tri-protic mineral acids and the polyhydroxy organic acids are present in the first aqueous phase in concentrations of from 0.01 mM to about 0.5 M, or preferably from about 5 mM to about 300 mM. When hydrochloric acid is used, it is present in lower amounts, from about 0.1 mM to about 50 mM, or preferably from about 0.5 mM to about 25 mM.

The lipid phase and first aqueous phase are mixed by mechanical turbulence, such as through use of rotating or vibrating blades, shaking, extrusion through baffled structures or porous pipes, by ultrasound, or by nozzle atomization, to produce a water-in-oil emulsion. Thus, bupivacaine or a salt thereof, such as bupivacaine phosphate, is encapsulated directly in the first step of MVL manufacture.

The whole water-in-oil emulsion is then dispersed into a second aqueous phase by means described above, to form solvent spherules suspended in the second aqueous phase. The term “solvent spherules” refers to a microscopic spheroid droplet of organic solvent, within which are suspended multiple smaller droplets of aqueous solution. The resulting solvent spherules therefore contain multiple aqueous droplets with the bupivacaine or a salt thereof, such as bupivacaine phosphate, dissolved therein. The second aqueous phase can contain additional components such as glucose, and/or lysine.

The volatile organic solvent is then removed from the spherules, for instance by surface evaporation from the suspension: When the solvent is substantially or completely evaporated, MVL are formed. Gases which can be used for the evaporation include nitrogen, argon, helium, oxygen, hydrogen, and carbon dioxide. Alternatively, the volatile solvent can be removed by sparging, rotary evaporation, or with the use of solvent selective membranes.

In some embodiments, an MVL is prepared in accordance with a process as described in U.S. Pat. No. 10,398,648, incorporated by reference herein in its entirety. In some embodiments, a MVL is prepared in accordance with a process as described in U.S. Pat. No. 9,585,838 incorporated by reference herein in its entirety.

In some embodiments, an MVL is prepared in accordance with a process as described in US Published Patent Applications US 2011-0250264, US 2013-0306759, US 2013-0177634, US 2013-0177633, US 2013-0177635, US 2013-0195965, US 2013-0177636, US 2013-0183373, US 2013-0177638, US 2013-0177637, US 2013-0183372, US 2013-0183375, US 2016-0361260 or US 2018-0092847, each of which is incorporated by reference herein in its entirety.

In some embodiments, an MVL is prepared in accordance with a process as described in U.S. Pat. Nos. 11,033,495; 11,179,336; 11,278,494; 11,304,904; 11,311,486; 11,357,727; 11,426,348; 11,452,691, each of which is incorporated by reference herein in its entirety.

In some embodiments, the multivesicular liposomes described herein can be combined, used in conjunction with, or used in an anesthetic or analgesic program with other anesthetics or analgesics.

Examples of anesthetics which can be administered according to the methods described herein, include but are not limited to, propofol, etomidate, methohexital and sodium thiopental, midazolam, diazepam, and ketamine, benzocaine, chloroprocaine, cocaine, cyclomethycaine, dimethocaine, propoxycaine, procaine, proparacaine, tetracaine, articaine, bupivacaine, carticaine, dibucaine, etidocaine, levobupivacaine, lidocaine, mepivacaine, piperocaine, prilocaine, ropivacaine, trimecaine, saxitoxin, and tetrodotoxin. Examples of amide anesthetics which can be administered according to the methods described herein, include but are not limited to, articaine, bupivacaine, carticaine, dibucaine, etidocaine, levobupivacaine, lidocaine, mepivacaine, piperocaine, prilocaine, ropivacaine, and trimecaine. In some embodiments, the multivesicular liposomes comprise bupivacaine, morphine, cytarabine, or their pharmaceutically acceptable salts as the therapeutic agent. In some embodiments, the multivesicular liposomes comprise bupivacaine phosphate, morphine sulfate, or cytarabine HCl.

The term “therapeutically effective” as it pertains to an anesthetic or a salt thereof present in the pharmaceutical compositions described herein, means that an anesthetic present in the first aqueous phase within the multivesicular liposome is released in a manner sufficient to achieve a particular level of anesthesia. Exact dosages will vary depending on such factors as the particular anesthetic, as well as patient factors such as age, sex, general condition, patient size, and the like. Those of skill in the art can readily take these factors into account and use them to establish effective therapeutic concentrations without resort to undue experimentation.

As used herein, “non-liposomal bupivacaine” refers to bupivacaine or a salt thereof that is not in liposomal form. For example, “non-liposomal bupivacaine” refers to bupivacaine or a salt thereof that is not comprised in a multivesicular liposome. The term “non-liposomal bupivacaine” encompasses compositions comprising bupivacaine, or a salt thereof, that is not in liposomal form.

Examples of analgesics can include opioid analgesics and non-opioid analgesics. Non-limiting examples of opioid analgesics include hydrocodone, oxycodone, propoxyphene, or fentanyl, thiosemicarbazone, p-nitrophenylhydrazone, o-methyloxime, semicarbazone, or bis (methylcarbamate), oxycodone, a pharmaceutically acceptable salt or its thiosemicarbazone, p-nitrophenylhydrazone, o-methyloxime, semicarbazone, or bis-methylcarbamate. Non-limiting examples of non-opioid analgesics useful in the present invention include aspirin; acetaminophen; a non-steroidal anti-inflammatory drug (NSAID), an arylalkanoic acid, a profen, a fenamic acid, an oxicam, a pyrazolidine derivative; a Cox-2 inhibitor, a local analgesic, an anti-depressant, an atypical analgesic, a psychotropic agent, an NMDA receptor antagonist, an α2-adrenoreceptor agonists and a synthetic drug having narcotic properties.

Embodiments of the present disclosure also include compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Academic Press, (Adeboye Adejareedit edit., 2020), hereby incorporated by reference in its entirety.

A pharmaceutically effective dose is that dose required to prevent, inhibit the occurrence, or treat (alleviate a symptom to some extent, preferably all of the symptoms) pain. One skilled in the art appreciates that compositions and methods of the present disclosure can be used to treat multiple types of pain, and that the effective dose may be different for different types of pain. Types of pain include but are not limited to thermal pain, chemical pain, inflammatory pain, ischemic pain, traumatic pain, blunt pain, sharp pain, prickling pain, and visceral pain. The pharmaceutically effective dose depends on the type of condition (e.g., pain), the composition used, the route of administration, the type of mammal being treated, the physical characteristics of the specific mammal under consideration (including but not limited to age, physical condition, surgical or other medical procedures being performed, circulatory capacity, cardiovascular function, pain tolerance, nerve function, liver function), concurrent medication, and other factors that those skilled in the medical arts will recognize.

In some embodiments the pharmaceutical composition comprises a therapeutically effective amount of bupivacaine phosphate. In some embodiments wherein the pharmaceutical composition comprises bupivacaine phosphate in an amount equivalent to from about 20 mg to about 300 mg of bupivacaine. In some embodiments the pharmaceutical composition comprises an amount equivalent to from about 133 mg to about 266 mg of bupivacaine.

In some embodiments the pharmaceutical composition comprises an amount equivalent to from about 10 mg to about 300 mg of bupivacaine. In some embodiments the pharmaceutical composition comprises an amount equivalent to from about 133 mg to about 266 mg of bupivacaine. In some embodiments the pharmaceutical composition comprises an amount equivalent to from about 10 mg to about 70 mg of bupivacaine. In some embodiments the pharmaceutical composition comprises an amount equivalent to from about 20 mg to about 60 mg of bupivacaine. In some embodiments the pharmaceutical composition comprises an amount equivalent to from about 20 mg to about 50 mg of bupivacaine. In some embodiments the pharmaceutical composition comprises an amount equivalent to from about 20 mg to about 40 mg of bupivacaine. In some embodiments the pharmaceutical composition comprises an amount equivalent to from about 20 mg to about 30 mg of bupivacaine. In some embodiments the pharmaceutical composition comprises an amount equivalent to from about 30 mg to about 60 mg of bupivacaine. In some embodiments the pharmaceutical composition comprises an amount equivalent to from about 30 mg to about 50 mg of bupivacaine. In some embodiments the pharmaceutical composition comprises an amount equivalent to from about 30 mg to about 40 mg of bupivacaine. In some embodiments the amount of the pharmaceutical composition described herein is equivalent to from about 10 mg to about 70 mg of bupivacaine. In some embodiments the amount of the pharmaceutical composition described herein is equivalent to from about 10 mg to about 60 mg of bupivacaine. In some embodiments the amount of the pharmaceutical composition described herein is equivalent to from about 20 mg to about 60 mg of bupivacaine. In some embodiments the amount of the pharmaceutical composition described herein is equivalent to from about 20 mg to about 50 mg of bupivacaine. In some embodiments the amount of the pharmaceutical composition described herein is equivalent to from about 20 mg to about 40 mg of bupivacaine. In some embodiments the amount of the pharmaceutical composition described herein is equivalent to from about 20 mg to about 30 mg of bupivacaine. In some embodiments the amount of the pharmaceutical composition described herein is equivalent to from about 30 mg to about 60 mg of bupivacaine. In some embodiments the amount of the pharmaceutical composition described herein is equivalent to from about 30 mg to about 50 mg of bupivacaine. In some embodiments the amount of the pharmaceutical composition described herein is equivalent to from about 30 mg to about 40 mg of bupivacaine.

In some embodiments, the amount of the pharmaceutical composition described herein is equivalent to 13.3 mg of bupivacaine. In some embodiments, the amount of the pharmaceutical composition described herein is equivalent to 26.6 mg of bupivacaine. In some embodiments, the amount of the pharmaceutical composition described herein is equivalent to 39.9 mg of bupivacaine. In some embodiments, the amount of the pharmaceutical composition described herein is equivalent to 53.2 mg of bupivacaine.

### Method of Administering an Injectate to a Lower Extremity of a Patient

Anesthetics of the present disclosure may be delivered regionally or locally. “Regional” or “local” anesthesia, as used herein, is distinct from general anesthesia and refers to anesthetic procedures which allow for the preferential delivery of an anesthetic to a specific region of the body, such as near a nerve or a nerve bundle. In contrast, general anesthesia allows for the systemic administration of an anesthetic, e.g., via intravenous administration. Regional or local anesthesia typically allows for a lower total body concentration (although elevated local concentrations) of an anesthetic to be administered to a subject for analgesia or diminished pain perception of at least a portion of the subject's body. For example, intrathecal anesthesia, epidural anesthesia, and nerve blocks are examples of regional or local anesthesia.

Provided herein are methods of administering an injectate, e.g., a pharmaceutical composition, to a lower extremity of a patient. The injectate can be used to treat pain, e.g., post-operative pain, in a subject in need thereof.

In some embodiments, an arthrotomy incision is performed to expose the knee capsule, and after retracting the medial aspect of the capsule, the adductor tubercle is palpated and an injection needle is inserted just proximal, for example 1 cm proximal, to the tubercle. The needle tip is advanced from the needle entry point to the injectate administration site. The needle tip advancement is typically along a plane between 10 and 20 degrees medial to the axis of the femur, and along a plane between 40 degrees and 70 degrees posterior to the femur, wherein the needle tip advancement is typically between 1 inch and 2 inches deep. In some embodiments, the needle is advanced until the hub of the needle touches or nearly touches the subject. Once at the injectate administration site, confirmation of the location of the needle tip can be optionally confirmed via fluoroscopy, for example live fluoroscopy. Once the needle tip is at the injectate administration site, injection can occur, wherein the injection can be of saline, sterile water, a pharmaceutical composition, or combinations thereof. The injection can include a single bolus injection or a split bolus injection. The injectate volume can be between approximately 1 ml and approximately 40 ml, including, but not limited to between approximately 1 ml and approximately 35 ml, approximately 5 ml and approximately 35 ml, approximately 10 ml and approximately 40 ml, approximately 10 ml and approximately 35 ml, approximately 10 ml and approximately 30 ml, approximately 15 ml and approximately 40 ml, approximately 15 ml and approximately 35 ml, approximately 15 ml and approximately 30 ml, approximately 15 ml and approximately 25 ml, or approximately 15 ml and approximately 20 ml. The injectate volume can be any specific amount between approximately 1 ml and approximately 40 ml, including approximately 1 ml, approximately 2 ml, approximately 3 ml, approximately 4 ml, approximately 5 ml, approximately 6 ml, approximately 7 ml, approximately 8 ml, approximately 9 ml, approximately 11 ml, approximately 12 ml, approximately 13 ml, approximately 14 ml, approximately 15 ml, approximately 16 ml, approximately 17 ml, approximately 18 ml, approximately 19 ml, approximately 20 ml, approximately 21 ml, approximately 22 ml, approximately 23 ml, approximately 24 ml, approximately 25 ml, approximately 26 ml, approximately 27 ml, approximately 28 ml, approximately 29 ml, approximately 30 ml, approximately 31 ml, approximately 32 ml, approximately 33 ml, approximately 34 ml, approximately 35 ml, approximately 36 ml, approximately 37 ml, approximately 38 ml, approximately 39 ml, or approximately 40 ml, or any partial volumes therein. In some embodiments, the volume of injectate is 20 ml, wherein the injectate can be administered as one bolus or two. In some embodiments, it is expected that the volume of injectate delivered stays within the area described for the intraarticular posteromedial landmark injection technique.

The bolus spreads to impact the saphenous nerve, the superficial saphenous nerves, the nerve to the vastus medialis, and the deep genicular nerves. The methods described herein are not adductor canal blocks, do not involve the adductor canal, but rather are distal to termination of the adductor canal. There are many advantages to the intraarticular posteromedial landmark injection technique described herein. The intraarticular posteromedial landmark injection methods described herein may be an efficient landmark alternative to ultrasound guided adductor canal block, wherein the surgeon may be able to deliver the intraarticular posteromedial landmark injection without additional assistance from an anesthesiologist. First, the surgeon-directed intraarticular posteromedial landmark injection can be performed without ultrasound guidance, simplifying administration of the methods. Further, an anesthesiologist often performs a nerve block in a different room with different hospital personnel from the location and hospital personnel of the underlying surgery. Thus, the intraarticular posteromedial landmark injection described herein can provide efficiencies in that the hospital does not need to coordinate locations or personnel for the intraarticular posteromedial landmark injection, because the intraarticular posteromedial landmark injection can be performed in the surgical theatre with the surgical personnel. An advantage of the novel intraarticular posteromedial landmark injection methods described herein is the surgeon is able to anesthetize both nerves typically involved in an adductor canal block (e.g., saphenous nerve, nerve to vastus medialis, and superficial saphenous nerves) as well as posterior knee capsule nerves (e.g., genicular nerves) with a single injection (i.e., one needle entry point, wherein the surgeon does not change needle angles or injectate administration sites during the injection. Therefore, the intraarticular posteromedial landmark injection is more efficient than other existing techniques because the person performing the injection does not need to position the injection needle for multiple needle entry points, and the person performing the injection does not need to re-position the needle to administer injectate to multiple injectate administration sites. In some embodiments, the intraarticular posteromedial landmark injection methods described herein can achieve sufficient analgesia without performing an “interspace between the popliteal artery and capsule of the posterior knee” (IPACK) procedure, or other posterior capsular block. Furthermore, an additional advantage of the novel intraarticular posteromedial landmark injection techniques described herein, are that patients subject to the intraarticular posteromedial landmark injection methods described herein do not experience quadriceps weakness. Without wishing to be bound by theory, it is believed that the intraarticular posteromedial landmark injection described herein does not contribute to quadriceps weakness because the injectate (e.g., any of the anesthesia injectates described herein) does not enter the adductor canal and/or travel proximately via the adductor canal. An additional advantage of the intraarticular posteromedial landmark injection methods disclosed herein is that the methods/technique is reproducible across patients with different body mass index (BMI) and/or different leg circumference.

The disclosure also includes pharmaceutical compositions for use in methods of administering a pharmaceutical composition to a leg of a patient for post-operative analgesia, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the sagittal plane of the femur and between approximately 40 degrees and 75 degrees posterior to the femur, wherein the needle is advanced a distance between one inch and two inches, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome. The disclosure also includes pharmaceutical compositions for use in a method of administering to a leg of a patient a pharmaceutical composition for post-operative analgesia, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the sagittal plane of the femur and between approximately 40 degrees and 75 degrees posterior to the femur, wherein the needle is advanced between one inch and two inches, wherein the pharmaceutical composition includes multivesicular liposomes containing bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and e) removing the organic solvent from the solvent spherules to form multivesicular liposomes encapsulating bupivacaine phosphate.

Further, the disclosure includes pharmaceutical compositions for use in a method of treating post-operative knee pain in a patient, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the sagittal plane of the femur and between approximately 40 degrees and 75 degrees posterior to the femur, wherein the needle is advanced a distance between one inch and two inches, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome. Additionally, the disclosure includes pharmaceutical compositions for use in a method of treating post-operative knee pain in a patient, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced a distance between one inch and two inches, wherein the pharmaceutical composition includes multivesicular liposomes containing bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and e) removing the organic solvent from the solvent spherules to form multivesicular liposomes encapsulating bupivacaine phosphate.

Additionally, the disclosure includes pharmaceutical compositions for use in a method of anesthetizing a subject in need of anesthetization following knee treatment, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome. The disclosure also includes pharmaceutical compositions for use in a method of anesthetizing a subject in need of anesthetization following knee treatment, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced a distance between one inch and two inches, wherein the pharmaceutical composition includes multivesicular liposomes containing bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and e) removing the organic solvent from the solvent spherules to form multivesicular liposomes encapsulating bupivacaine phosphate.

Additionally, the disclosure includes pharmaceutical compositions for use in a method of reducing knee pain in a subject, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome. The disclosure also includes pharmaceutical compositions for use in a method of reducing knee pain in a subject, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition includes multivesicular liposomes containing bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and c) removing the organic solvent from the solvent spherules to form multivesicular liposomes encapsulating bupivacaine phosphate.

Additionally, the disclosure includes pharmaceutical compositions for use in a method of reducing post-operative knee pain in a subject, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome. The disclosure also includes pharmaceutical compositions for use in a method of reducing post-operative knee pain in a subject, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition includes multivesicular liposomes containing bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and e) removing the organic solvent from the solvent spherules to form multivesicular liposomes encapsulating bupivacaine phosphate.

Additionally, the disclosure includes pharmaceutical compositions for use in a method of anesthetizing genicular nerves, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome. The disclosure also includes pharmaceutical compositions for use in a method of anesthetizing genicular nerves, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition includes multivesicular liposomes containing bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and c) removing the organic solvent from the solvent spherules to form multivesicular liposomes encapsulating bupivacaine phosphate.

Additionally, the disclosure includes pharmaceutical compositions for use in a method of anesthetizing the saphenous nerve and nerve to vastus medialis, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome. The disclosure also includes pharmaceutical compositions for use in a method of anesthetizing the saphenous nerve and nerve to vastus medialis, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition includes multivesicular liposomes containing bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and e) removing the organic solvent from the solvent spherules to form multivesicular liposomes encapsulating bupivacaine phosphate.

Additionally, the disclosure includes pharmaceutical compositions for use in a method of anesthetizing a lower extremity, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome. The disclosure also includes pharmaceutical compositions for use in a method of anesthetizing a lower extremity, the method comprising: selecting an entry point of an injection needle in a leg of a patient; inserting the injection needle into the patient at the needle entry point; advancing the tip of the injection needle to an injectate administration site; and administering a pharmaceutical composition at the injectate administration site; wherein the needle entry point is proximal to the adductor tubercle, and wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches, wherein the pharmaceutical composition includes multivesicular liposomes containing bupivacaine or a salt thereof; phosphoric acid; a lipid component comprising at least one amphipathic lipid and at least one neutral lipid lacking a hydrophilic head group; and, optionally, a cholesterol and/or a plant sterol, wherein said multivesicular liposomes are made by a process comprising: a) preparing a first aqueous component comprising phosphoric acid; b) preparing a lipid component comprising at least one organic solvent, at least one amphipathic lipid, and at least one neutral lipid lacking a hydrophilic head group; c) mixing said first aqueous component and said lipid component to form a water-in-oil emulsion, wherein at least one component comprises bupivacaine or a salt thereof; d) mixing said water-in-oil emulsion with a second aqueous component to form solvent spherules; and e) removing the organic solvent from the solvent spherules to form multivesicular liposomes encapsulating bupivacaine phosphate.

### EXAMPLES

The invention is further described in the following examples, which do not limit the scope of the invention described in the claims.

### Example 1: Human Cadaver Lab Legs

The intraarticular posteromedial landmark injection methods described herein were performed on five human cadaver legs at the Pacira Innovation & Training Center in April 2023. The results of the five procedures are detailed below.

Leg 1: Specimen characteristics: 80-year-old male. Left leg. BMI=24.37. Thigh circumference=47.5 cm. An ultrasound-guided adductor canal (AC) block was performed with 20 mL of solution (sterile saline+green dye) targeting 10 mL at the saphenous nerve and 10 mL at the nerve to vastus medialis (NVM). Following the ultrasound-guided AC block, an arthrotomy incision was made to expose the knee capsule. After retracting the medial aspect of the capsule, the adductor tubercle was palpated and just proximal to the tubercle, a 4 inch, 18 gauge, Tuohy needle was inserted 20 degrees medial to the axis of the femur and 30 degrees posterior to the femur and the needle was advanced to the 6 cm mark. Once the needle position was confirmed via fluoroscopy, 10 mL of combined solution (sterile water, omnipaque, and blue dye) was injected under live fluoroscopy. The needle was left in place and after a pause, an additional 10 mL of the same solution was injected under live fluoroscopy. Fluoroscopic images demonstrated spread of injectate, 15 cm proximally from the joint line. Blue pigmentation was noted on the medial aspect of the thigh through the skin post-injection.

Upon dissection, the needle tip from the intraarticular posteromedial landmark injection appeared to be in the subcutaneous space superficial to the vastus medialis. There was no blue staining from the intraarticular posteromedial landmark injection found within the adductor canal. There was no staining of blue dye between the vastus medialis and sartorius muscles but rather only in the subcutaneous tissue. There was green staining from the ultrasound-guided adductor canal block within the adductor canal that stained the saphenous nerve approximately 7.5-9.5 cm down the entire length of the saphenous nerve, terminating at the femoral triangle along with staining of the nerve to vastus medialis. The infrapatellar saphenous was found to be superficial to the sartorius along its entire course and was not stained by either the green (ultrasound-guided Adductor Canal block) or blue (intraarticular posteromedial landmark injection described herein) dyes.

Leg 2: Specimen characteristics: 81-year-old Female. Left leg. BMI=26.45. Thigh circumference=47.5. An ultrasound-guided block of the anterior femoral cutaneous nerve (AFCN) was performed with 10 mL of solution (sterile water+green dye). Once the AFCN block was completed, a left knee arthrotomy was performed to expose the knee capsule. From the inside of the knee capsule, the adductor tubercle was palpated and just proximal to the tubercle, and a 1.5 inch, 18 gauge hypodermic needle was inserted 20 degrees medial to the axis of the femur and 40 degrees posterior to the femur. The entire 1.5-inch needle was inserted. Once the needle position was confirmed via fluoroscopy, 10 mL of combined solution (sterile water, omnipaque, and blue dye) was injected under live fluoroscopy. The needle was left in place and after a pause, an additional 10 mL of the same solution was injected under live fluoroscopy. Fluoroscopic images demonstrated spread of injectate 9.5 cm proximal to from the joint line.

Upon dissection, heavy blue staining from the intraarticular posteromedial landmark injection was observed in the interval between the vastus medialis muscle and the subcutaneous tissue. Blue staining of the adductor tendon, and hamstrings tendon of adductor magnus was also observed, with some staining noted in the interval between the vastus medialis and the adductor tendon. There was significant blue staining of the distal portion of the infrapatellar branch of the saphenous nerve as well as posteromedial staining of the posterior capsule. The AFCN branches, as well as the infrapatellar saphenous, were observed to come off a common trunk of the anterior division of the femoral nerve separate from the posterior division and the saphenous proper.

The sartorius muscle was dissected and followed down to its distal attachment at the pes anserinus on Gerdy's tubercle of the tibia. Blue staining was well confined in the interval between the pes and the capsule, and it is through that the superomedial genicular branches would be covered by this procedural approach. The tendon of the semimembranosus was unstained; however, the area deep to the tendon of the semimembranosus was well stained. There was no blue staining of the sciatic nerve in the region of the popliteal fossa. The posterior capsule was stained with blue dye all the way to the lateral condyle.

There was no blue staining within the adductor canal as the staining remained distal to the adductor canal. The green dye stained three branches of the AFCN which were located superficial to the fascial lata and deep to the subcutaneous tissue.

Leg 3: Specimen characteristics: 73-year-old Male. Right leg. BMI=24.68. Thigh circumference=49.5. A right knee arthrotomy was performed to expose the capsule of the knee. From the inside of the knee capsule, the adductor tubercle was palpated and 1 cm proximal to the tubercle, a 1.5 inch, 18 gauge hypodermic needle was inserted 10 degrees medial to the axis of the femur and 55 degrees posterior with all 1.5 inches of the needle advanced. Once the needle position was confirmed via fluoroscopy, 10 mL of combined solution (sterile water, omnipaque, and blue dye) was injected under live fluoroscopy. The needle was left in place and after a pause, an additional 10 mL of the same solution was injected under live fluoroscopy. Fluoroscopic images demonstrated spread of injectate approximately 12.5 cm proximally from the joint line. Next, an ultrasound-guided adductor canal block, using a trans-sartorial approach, was performed using 20 mL of green dye solution (sterile water+green dye).

Upon dissection, blue dye staining from the intraarticular posteromedial landmark injection was observed between vastus medialis and the adductor tendon. Most of the blue staining was located posterior to the inter-muscular fascial septum that separates the anterior compartment from the medial compartment, keeping the dye in the medial and posterior compartments of the knee. Some blue dye spread was noted in the very distal aspect of the adductor canal through the hiatus from the inferior border of the adductor canal to meet the diffusion of the green dye from the ultrasound-guided adductor canal block which was maintained in the adductor canal.

Leg 4: Specimen characteristics: 83-year-old Male. Left leg. BMI=22.24. Thigh circumference=39.0 cm. A right knee arthrotomy was performed to expose the capsule of the knee. From the inside of the knee capsule, the adductor tubercle was palpated and 1 cm proximal to the tubercle, a 1.5 inch, 18 gauge hypodermic needle was inserted 10 degrees medial to the axis of the femur and approximately 30 degrees posterior with all 1.5 inches of the needle advanced. Once the needle position was confirmed via fluoroscopy, 20 mL of combined solution (sterile water, omnipaque, and blue dye) was injected under live fluoroscopy. Fluoroscopic images demonstrated spread of injectate approximately 11.5 cm proximally from the joint line. An ultrasound-guided block was not performed on this specimen.

Upon dissection, the infrapatellar branch of the saphenous nerve demonstrated where it pierces the sartorius muscle proximally and stays superficial to the sartorius muscle. Blue dye staining from the intraarticular posteromedial landmark injection was observed between the vastus medialis and sartorius muscles. Blue dye staining did not extend beyond the intermuscular septum in this specimen. The posterior capsule was not stained.

Leg 5: Specimen characteristics: 73-year-old Male. Left leg. BMI=24.68. Thigh circumference=49.5. A left knee arthrotomy was performed to expose the left knee capsule. From the inside of the knee capsule, the adductor tubercle was palpated and just proximal to the tubercle, a 1.5 inch, 18 gauge hypodermic needle was inserted approximately 10 degrees medial to the axis of the femur and 70 degrees posterior with all 1.5 inches of the needle advanced. Once the needle position was confirmed via fluoroscopy, 20 mL of combined solution (sterile water, omnipaque, and blue dye) was injected under live fluoroscopy. An ultrasound guided block was not performed on this specimen. Upon dissection, the adductor magnus tendon was identified and blue dye staining was observed anterior to the adductor magnus tendon. Significant posterior capsule staining was noted. Blue dye staining was not observed in the intermuscular septum or the sartorius space.

### Example 2: Summary of Human Cadaver Lab Results

TABLE
Dye Injection Procedures and Structures Dyed for Each Cadaver

| | Surgeon-administered characteristics | | | | Structures dyed | |
|---|---|---|---|---|---|---|
| | | Needle | Time | | Green | Blue |
| | Needle | angle, ° | between | Location of the | (US-guided | (Surgeon- |
| | length, | posterior | injections, | ISN (relative to | ACB or | administered |
| Cadaver | inches* | to femur | min | sartorius muscle) | AFCNB)^{†} | injection) |
| 1 | 4 | 30 | 9 | Superficial | SN | None |
| | | | | | NVM | |
| | | | | | Adductor canal | |
| 2 | 1.5 | 40 | 5 | Superficial | AFCN branches | SN |
| | | | | | | ISN |
| | | | | | | NVM |
| | | | | | | Posterior capsule |
| | | | | | | Genicular nerves^{‡} |
| 3 | 1.5 | 55 | 5 | Within the AC | SN | SN |
| | | | | | NVM | ISN |
| | | | | | Adductor canal | NVM |
| | | | | | | Posterior capsule |
| | | | | | | Genicular nerves^{‡} |
| 4^{§} | 1.5 | 30 | 0 | Superficial | NA | None |
| 5^{§} | 1.5 | 70 | 0 | Within the AC | NA | SN |
| | | | | | | ISN |
| | | | | | | NVM |
| | | | | | | Genicular nerves* |

*All needles were 18-gauge sharp.
^{†}A US-guided ACB was performed in cadavers 1 and 3; a US-guided AFCNB was performed in cadaver 2.
^{‡}Staining of genicular nerves was inferred based on proximity, but genicular nerves were not formally dissected.
^{§}Only landmark approach/methylene blue was administered in cadavers 4 and 5.
ACB, adductor canal block; AFCNB, anterior femoral cutaneous nerve block; ISN, infrapatellar branch of the saphenous nerve; NVM, nerve to the vastus medialis muscle; SN, saphenous nerve; US, ultrasound.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of reducing knee pain in a subject, the method comprising:
a) selecting an entry point of an injection needle in a subject's leg;
b) inserting the injection needle into the needle entry point;
c) advancing the tip of the injection needle to an injectate administration site; and
d) administering a pharmaceutical composition at the injectate administration site;
wherein the needle entry point is proximal to the adductor tubercle, and
wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches,
thereby reducing knee pain in a subject.

2. The method of claim 1, wherein the pharmaceutical composition comprises multivesicular liposomes.

3. The method of claim 2, wherein the multivesicular liposomes comprise:
bupivacaine or a salt thereof;
phosphoric acid;
a lipid component comprising at least one amphipathic lipid and at least one neutral lipid lacking a hydrophilic head group; and,
optionally, a cholesterol and/or a plant sterol,
wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

4. The method of claim 1, wherein the injection needle is 1.5 inches long.

5. The method of claim 4, wherein the injection needle tip is advanced a distance of about 1.5 inches along the angle.

6. The method of claim 1, wherein the injection needle is an 18 gauge bevel needle.

7. The method of claim 1, wherein selecting an entry point comprises palpating the adductor tubercle.

8. The method of claim 7, wherein the entry point is approximately 1 cm from the adductor tubercle at the proximal border of the adjacent femoral condyle.

9. The method of claim 1, wherein the administering comprises administering about 20 mL of the pharmaceutical composition in a single bolus.

10. The method of claim 1, wherein the administering comprises administering about 20 mL of the pharmaceutical composition in a split bolus.

11. The method of claim 10, wherein the split bolus comprises two boluses wherein each bolus is administered without repositioning the injection needle.

12. The method of claim 10, wherein the split bolus comprises administering approximately a 10 mL bolus of the pharmaceutical composition, followed by a rest period, followed by approximately a 10 mL bolus of the pharmaceutical composition.

13. The method of claim 1, further comprising performing a knee arthrotomy to expose the knee capsule prior to selecting the entry point of the injection needle.

14. The method of claim 1, wherein the pharmaceutical composition spreads to affect two or more of: the saphenous nerve, the superficial branch of the saphenous nerve, the nerve to the vastus medialis, and the deep genicular nerves.

15. The method of claim 14, wherein the pharmaceutical composition spreads to affect three or more of: the saphenous nerve, the superficial branch of the saphenous nerve, the nerve to the vastus medialis, and the deep genicular nerves.

16. The method of claim 15, wherein the pharmaceutical composition spreads to affect the saphenous nerve, the superficial branch of the saphenous nerve, the nerve to the vastus medialis, and the deep genicular nerves.

17. The method of claim 1, wherein less than 10% of the pharmaceutical composition is detectable in the adductor canal.

18. The method of claim 1, wherein less than 10% of the pharmaceutical composition is detectable around the anterior femoral or lateral femoral cutaneous nerves.

19. A method of treating post-operative knee pain in a subject, the method comprising:
a) selecting an entry point of an injection needle in a subject's leg;
b) inserting the injection needle into the needle entry point;
c) advancing the tip of the injection needle to an injectate administration site; and
d) administering a pharmaceutical composition at the injectate administration site;
wherein the needle entry point is proximal to the adductor tubercle, and
wherein the advancing the tip of the injection needle utilizes an angle of between approximately 10 and approximately 20 degrees medial to the axis of the femur and between approximately 40 degrees and 75 degrees posterior to the femur, wherein the needle is advanced between one inch and two inches,
thereby treating post-operative knee pain in the subject.

20. The method of claim 19, wherein the pharmaceutical composition comprises multivesicular liposomes.

21. The method of claim 20, wherein the multivesicular liposomes comprise:
bupivacaine or a salt thereof;
phosphoric acid;
a lipid component comprising at least one amphipathic lipid and at least one neutral lipid lacking a hydrophilic head group; and,
optionally, a cholesterol and/or a plant sterol,
wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

22. The method of claim 19, wherein the injection needle is 1.5 inches long.

23. The method of claim 22, wherein the injection needle tip is advanced a distance of about 1.5 inches along the angle.

24. The method of claim 19, wherein the injection needle is an 18 gauge bevel needle.

25. The method of claim 19, wherein selecting an entry point comprises palpating the adductor tubercle.

26. The method of claim 25, wherein the entry point is approximately 1 cm from the adductor tubercle at the proximal border of the adjacent femoral condyle.

27. The method of claim 19, wherein the administering comprises administering about 20 mL of the pharmaceutical composition in a single bolus.

28. The method of claim 19, wherein the administering comprises administering about 20 mL of the pharmaceutical composition in a split bolus.

29. The method of claim 28, wherein the split bolus comprises two boluses wherein each bolus is administered without repositioning the injection needle.

30. The method of claim 28, wherein the split bolus comprises administering approximately a 10 mL bolus of the pharmaceutical composition, followed by a rest period, followed by approximately a 10 mL bolus of the pharmaceutical composition.

31. The method of claim 19, further comprising performing a knee arthrotomy to expose the knee capsule prior to selecting the entry point of the injection needle.

32. The method of claim 19, wherein the pharmaceutical composition spreads to affect two or more of: the saphenous nerve, the superficial branch of the saphenous nerve, the nerve to the vastus medialis, and the deep genicular nerves.

33. The method of claim 32, wherein the pharmaceutical composition spreads to affect three or more of: the saphenous nerve, the superficial branch of the saphenous nerve, the nerve to the vastus medialis, and the deep genicular nerves.

34. The method of claim 32, wherein the pharmaceutical composition spreads to affect the saphenous nerve, the superficial branch of the saphenous nerve, the nerve to the vastus medialis, and the deep genicular nerves.

35. The method of claim 19, wherein less than 10% of the pharmaceutical composition is detectable in the adductor canal.

36. The method of claim 19, wherein less than 10% of the pharmaceutical composition is detectable around the anterior femoral or lateral femoral cutaneous nerves.

37. A method of reducing knee pain in a subject, the method comprising:
a) selecting an entry point of an injection needle in a subject's leg;
b) inserting the injection needle into the needle entry point;
c) advancing the tip of the injection needle to an injectate administration site; and
d) administering a pharmaceutical composition at the injectate administration site;
wherein the needle entry point is proximal to the adductor tubercle, and
wherein the advancing the tip of the injection needle utilizes an angle of between approximately 10 and approximately 20 degrees medial to the axis of the femur and between approximately 40 degrees and 75 degrees posterior to the femur, wherein the needle is advanced a distance between one inch and two inches along the angle,
wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

38. A method of treating post-operative knee pain in a subject, the method comprising:
a) selecting an entry point of an injection needle in a subject's leg;
b) inserting the injection needle into the needle entry point;
c) advancing the tip of the injection needle to an injectate administration site; and
d) administering a pharmaceutical composition at the injectate administration site;
wherein the needle entry point is proximal to the adductor tubercle, and
wherein the advancing the tip of the injection needle utilizes an angle of between approximately 10 and approximately 20 degrees medial to the axis of the femur and between approximately 40 degrees and 75 degrees posterior to the femur, wherein the needle is advanced a distance between one inch and two inches along the angle,
wherein the pharmaceutical composition comprises: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.
